(19) United States
(12) Patent Application Publication
(10) Pub. No.: US 2003/0014038 A1
(43) Pub. Date: Jan. 16, 2003

(54) Article and method of use for absorbing bodily fluids

(76) Inventor: Bruce Fine, Wayne, NJ (US)

Correspondence Address: Patrick P. Zaretski, Esq., Law Offices, 75 Carol Place, Wayne, NJ, 07470-2925, US

(21) Appl. No.: 09/850,497

(22) Filed: May 7, 2001

(51) Int. Cl. A61F 13/15; A61F 13/20; A61M 1/00

(52) U.S. Cl. 604/540; 604/360; 604/368; 604/378; 604/385.101

(57) ABSTRACT

A disposable superabsorbent article, and methods of use thereof, has superabsorbent cross-linked acrylate particles enveloped in a clean or sterile environment by an adhesively-sealed or heat-sealed plastic; paper; creped or microcreped paper or plastic; or woven, netted, nonwoven or fabric envelope, in shapes allowing for internal or external use, which permit the improved absorption and retention of fluids in medical, dental, veterinary, emergency care, mortuary, or surgical settings. Axial, circumferential, stellate, or other organized or random pleating or creping in either a single layer or in strata, and breakaway tabs in an alternative embodiment, are provided in the envelope to allow for controlled directional expansion of the fluid absorption. The superabsorbent article may be impregnated with therapeutically-effective amounts of antibiotic, antimicrobial, anticoagulating, or hemostatic or other agents.

### FIELD OF THE INVENTION

The present invention relates to disposable absorbent articles for use in the practical health sciences, and more particularly to a superabsorbent article, and methods of use thereof, which allows for the improved absorption and retention of fluids in medical, dental, veterinary, emergency care, mortuary, or surgical settings.

### BACKGROUND OF THE INVENTION

There is generally described in the art disposable articles of manufacture which may be used for the absorbance and retention of body fluids in the practical health sciences, namely bandages, pressure pads, gauze applications, tampons, and the like. The products incorporate an absorbent batt which is used to absorb and retain or contain body fluids. Initially, in many of these products, especially diapers and sanitary napkins, the absorbent batt comprised what is termed “wadding” or plies of tissue, types of tissue including, among other materials, cellulose or woodpulp. The wadding was disposed between a liquid-impermeable backing and a liquid-permeable facing and the plies of tissue were used to absorb and, hopefully, contain the liquid within the product. As this was only a physical absorption and retention of water, however, there was only a limited finite capacity of the batt's absorbance and retention properties, and often there would be leakage from the absorbent article.

However, chemical superabsorption of such fluids is also possible, and this phenomenon provides for better fluid absorption and retention. For example, U.S. Pat. No. 3,669,103 to Harper et al. teaches the use of tightly cross-linked water-swellable, water-insoluble polymers, such as polyacrylates, hydrolyzed polyacrylamides, and copolymers of acrylamide with acrylic acid or an alkali metal salt like sodium acrylate, contained in a body-conforming support such as a bandage, diaper, or tampon. However, this patent fails to teach use of the alkali metal salt as the sole superabsorbent material used in the superabsorbent article. It also fails to teach both use of the article in body cavities as well as on the surface of the animal's body and the inclusion of various other medicinal agents which may be usefully impregnated upon or within the article. Finally, the patent fails to teach a covering that allows for directional control of the expansion of the article.

Additionally, Japanese Patent JP407163648A to Hashimoto et al. teaches a liquid absorbent and absorbent article formed out of either an N-vinylacetamide homopolymer, or a cross-linked copolymer thereof with a monomer such as sodium acrylate, which ensures high diffusivity at a liquid absorbing process and is therefore useful for various types of products such as a bandage, diaper, or tampon. Again, however, this patent fails to teach use of the alkali metal acrylic salt as the sole superabsorbent material used in the superabsorbent article. It also fails to teach both use of the article in body cavities as well as on the surface of the animal's body and the inclusion of various medicinal agents which may be usefully impregnated upon or within the article. Finally, the patent fails to teach a covering that allows for directional control of the expansion of the article.

Finally, Japanese Patent JP405068694A to Sakai et al. teaches an absorbent article in which absorbent paper coated or impregnated with an antimicrobial silica gel and acrylic resin on the surface or inner side of the absorber is arranged so that the absorbent article has a front liquid-permeable surface, a rear liquid-impermeable surface, and absorbent material provided therebetween. Yet again, however, this patent fails to teach use of the alkali metal acrylic salt as the sole superabsorbent material used in the superabsorbent article. It also fails to teach both use of the article in body cavities as well as on the surface of the animal's body and the inclusion of various other medicinal agents which may be usefully impregnated upon or within the article. Finally, the patent fails to teach a covering that allows for directional control of the expansion of the article.

In the practical health sciences or mortuary setting, it is desirable to have directional control over the expansion of the absorbent article because this allows for uniform conformation of the article along the area where fluids are elaborated from, especially important when dealing with a closed and/or defined body cavity. Especially in the surgical or medical fields, such conformation allows for increased pressure transmitted along this area to minimize or stop hemorrhage, the uniform application or impregnation of various medicaments found on or within the absorbent article, protection of nearby or surrounding tissue or structures, and later easier removal of the article from this elaborative area.

In the surgical setting, it is also often desirable to have a superabsorbent padding contained within a hydrophobic covering with a hydrophilic cutout in the shape of the organ or vessel being operated on or one close to such an organ or vessel. This padding permits direct measurement and containment of finite quantities of blood or other fluid elaborated during the surgical procedure, allows for a clearer field of vision and facilitated expansion of the organ or vessel within the body cavity, and allows for minimum leakage of fluid into adjacent body cavities or organs.

The application or impregnation of various medicaments found on or within the absorbent article is also desirable because this allows for direct and uniform delivery of these medicaments to an actively bleeding or elaborating site, thereby lessening the transit time of these agents versus other such delivery systems as ingestion or injection.

Consequently, in view of the limitations of the prior art, it would be desirable to provide a superabsorbent article with improved absorption and retention of fluids in medical, dental, veterinary, emergency care, or surgical settings which is of low cost to manufacture; may be used either on an animal's body surface or internally in body cavities or other areas; allows for directional control of expansion during fluid absorption and retention; and which may be impregnated with various useful medicinal agents either on the surface of or within the article.

### OBJECTS OF THE INVENTION

Accordingly, it is an object of the present invention to provide a superabsorbent article for the improved absorption and retention of fluids, and methods of use thereof, wherein the superabsorbent material is acrylic acid or cross-linked alkali metal salt particles thereof incorporated within the article.

It is an additional object of the present invention to provide for a superabsorbent article for the improved absorption and retention of fluids, and methods of use thereof, wherein the absorbent core is enveloped by an adhesively-sealed or heat-sealed plastic; paper; creped or microcreped paper or plastic; or woven netted, nonwoven, or fabric envelope in shapes allowing for internal or external use, having axial, circumferential, stellate, or other organized or random pleating, in either a single layer or in strata, and which may be sterilized to prevent infection, to provide for directionally-controlled expansion of the article during fluid absorption and retention.

It is an additional object of the present invention to provide for a superabsorbent article for the improved absorption and retention of body fluids, and methods of use thereof, wherein therapeutically-effective amounts of antibiotic, antimicrobial, anticoagulating, or hemostatic agents or other drugs are impregnated either on the surface of or within the superabsorbent article.

### SUMMARY OF THE INVENTION

Consequently, to achieve these aims and objectives, the present invention provides for a disposable superabsorbent article, and methods of use thereof, in which superabsorbent cross-linked sodium acrylate particles are enveloped in a clean or sterile environment by an adhesively-sealed or heat-sealed plastic; paper; creped or microcreped paper or plastic; or woven, netted, nonwoven or fabric envelope, in shapes allowing for internal or external use, which permit the improved absorption and retention of fluids in medical, dental, veterinary, emergency care, mortuary, or surgical settings. Axial, circumferential, stellate, or other organized or random pleating or creping, in either a single layer or in strata, are provided in the covering to allow for controlled directional expansion of the fluid absorption. In one embodiment of the invention, breakaway tabs are provided on the ends of the superabsorbent article to permit for additional absorption of fluids. In another embodiment of the invention, the superabsorbent article is flattened and shaped to conform to a particular animal organ to facilitate fluid collection during surgery. The superabsorbent article may be sterilized to prevent infection, and the pleats may be impregnated with therapeutically-effective amounts of antibiotic, antimicrobial, anticoagulating, or hemostatic or other agents.

### BRIEF DESCRIPTION OF THE DRAWINGS

The drawings shall be added later by amendment.

### DETAILED DESCRIPTION OF THE DRAWINGS

There is generally illustrated a superabsorbent article of a size and conformation which allows for easy application or placement on the external surface or a body cavity of an animal. The article consists of a superabsorbent core wherein superabsorbent particles, for example cross-linked particles of acrylic acid, preferably in the form of sodium acrylate but encompassing any alkali metal salt of acrylic acid, are contained within a sterile, tightly-sealed envelope of material. Cross-linking may be performed by the methods described in U.S. Pat. No. 3,669,103 to Harper et al., which is herein incorporated by reference. However, there is no preferred method of cross-linking required for this invention. The envelope material can be plastic, with the plastic heat-sealed, symmetrically or asymmetrically point-sealed, or otherwise adhesively-sealed, along either the entire outer edge of the perimeter of the envelope or sealed only at the two lateral ends of the envelope. Additional materials which may be used to construct the envelope, which may be sealed along either the entire outer edge of the perimeter of the envelope or sealed only at the two lateral ends of the envelope, include paper; creped or microcreped paper; or woven, netted, nonwoven, or fabric envelope. Plastic may also be provided along only one side of the article, and the rest encapsulated by a choice of the remaining materials, to prevent cutting by sharp instruments during a surgical or other procedure.

Formed from the envelope material along the perimeter of the article, pleats or creping can be found along the article's perimeter which allow for directional control of the expansion of the article as fluids are absorbed and retained. The pleats allow for easy storage of the article in a sterile wrapper or other container yet permit an increased surface-to-volume ratio for expansion and retention of fluids. These pleats may themselves be previously sterilized to prevent infection upon use with a patient. Additionally, these pleats permit conformation to the dimensions of any body cavity the article may be placed into, which in turn allows for pressure to minimize hemorrhage after surgery or injury, displacement of any interfering structures within the body cavity, later easy removal of the article, etc. These pleats are disposed axially, circumferentially, in a stellate pattern, or in an otherwise organized or random pattern, in either a single layer along the surface of the article or in strata disposed throughout the body of the article, for controlled expansion along the three dimensions of the article. The pleating strata therefore may extend further internally to the inner perimeter of the article into the superabsorbent core in order to increase the surface-to-volume ratio available for expansion. Pleating or creping may also take the form of a “teabag” fold along only the outer perimeter of the article's surface. Other conformations of pleatings may become obvious to those skilled in the art, however, and the examples mentioned here are not meant to limit the embodiment of the present invention in any manner other than those present in the claims.

Additionally, breakaway tabs may be disposed at various locations, especially at the ends, of the superabsorbent article in order to further increase the surface-to-volume ratio of the article and permit increased absorption and retention capacities of the article.

In an alternative embodiment of the present invention, to better facilitate the absorption, retention, and collection of fluids during an internal surgical procedure, the superabsorbent article may be flattened and corresponding in shape and size to various animalian internal organs, such as kidney-shaped for the kidneys, oval for the stomach, roughly triangular for the lungs, and so on. Flattening the article will allow for easy placement within the thoracic and abdominal cavities, while the various forms of pleating or creping will still allow for directional control of the expansion of the article.

The superabsorbent article may be impregnated with therapeutically-effective amounts of antibiotic, antimicrobial, anticoagulating, or hemostatic or other agents, either each separately or in combinations thereof. For example, the antimicrobial agents triclosan, silver chlorhexidine, glutaraldehyde, iodine, or ethanol can be deposited onto or within any or all of the layers of the article. All layers may be so impregnated with the antimicrobial agent to sanitize the environment or cavity in which the article is placed, or only the internal layers adjacent to the superabsorbent core, to maintain or convert any absorbed fluids into a sanitized state. Conversely, only the external layers of the article may be so impregnated, so that the absorbed, retained liquids are not sanitized and therefore may be sampled, tested, or retained for other purposes.

Anticoagulant agents may be used to permit free bleeding out of an infected wound site or surgically-opened cavity, also permitting blood to pool into the internal layers of the article before coagulating and thereby forming a barrier against penetration of other fluids. Hemostatic agents can retard or stop bleeding from active wound sites either externally or within body cavities, while still absorbing such blood or other fluids as have already leaked from the tissue. Antibiotics or other pharmaceutical compounds could be included to deliver such agents directly into an open wound site or body cavity without need to go through a “first-pass” effect as seen in ingestion of such drugs or using injection.

It is to be understood that the use of the term “animal” throughout this application is meant to encompass human beings. Furthermore, while the present invention has been described in connection with exemplary embodiments thereof, it will be understood that many modifications in both design and use will be apparent to those of ordinary skill in the art; and this application is intended to cover any adaptations or variations thereof. Therefore, it is manifestly intended that this invention be only limited by the claims and the equivalents thereof.

The invention to be claimed is:

1. A superabsorbent article to be used in the absorption and retention of fluids, comprising:
a core of liquid-swellable cross-linked superabsorbent particles, and
a liquid-permeable envelope in which said superabsorbent core is situated,
said envelope sealed along the outer perimeter,
said envelope further having pleats or creping formed from the material from which said envelope is constructed,
said pleats or creping running axially, circumferentially, in a stellate pattern, or in random or other organized patterns,
said patterns being disposed in the envelope in at least one layer of pleats or creping along the perimeter thereof.

2. The article of claim 1, wherein said sealed envelope is sterilized.

3. The article of claim 1, wherein said superabsorbent particles are acrylic acid.

4. The article of claim 3, wherein said superabsorbent particles are an alkali metal salt of acrylic acid.

5. The article of claim 1, wherein the envelope is constructed from a material selected from a group consisting of plastic, paper, creped or microcreped paper or plastic, woven, netting, or unwoven or other fabric.

6. The article of claim 5, wherein the envelope is constructed from plastic along one surface of the article and also a material selected from one of the remaining group members along the article's other surfaces.

7. The article of claim 5, wherein the envelope is sealed at symmetric points.

8. The article of claim 5, wherein the envelope is sealed at asymmetric points.

9. The article of claim 5, wherein the envelope is sealed at only the lateral ends of the outer perimeter.

10. The article of claim 1, further comprising at least one breakaway tab detachably affixed to the outer perimeter of the envelope.

11. The article of claim 1, further comprising a therapeutically-effective amount of at least one physiologically-active agent impregnated onto or within the article selected from a group consisting of antibiotic agents, antimicrobial agents, anticoagulating agents, hemostatic agents, or other pharmaceutical compounds.

12. The article of claim 1, wherein the envelope is flattened and shaped analogously to animalian internal organs, cavities, or vessels, or any portion of these thereof.

13. A method of using a superabsorbent article according to claim 1 in a medical, dental, surgical, mortuary, or veterinary procedure.
